(19) United States
(12) Patent Application Publication
(10) Pub. No.: US 2025/0135119 A1
(43) Pub. Date: May 1, 2025

(54) FLOW CONTROL DEVICES FOR CANCER TREATMENT SYSTEMS

(71) Applicant: Boston Scientific Scimed, Inc., Maple Grove, MN (US)

(72) Inventors: Ajay Gupta, Minneapolis, MN (US); Randy Raymond, Maple Grove, MN (US); Mark Steven Smith, Coon Rapids, MN (US)

(21) Appl. No.: 18/921,475

(22) Filed: Oct. 21, 2024

Related U.S. Application Data
(60) Provisional application No. 63/546,367, filed on Oct. 30, 2023.

(51) Int. Cl. A61M 5/315; A61M 5/145

(52) U.S. Cl. CPC A61M 5/31528; A61M 5/1452; A61M 2202/0007; A61M 2202/04; A61M 2205/3334; A61M 2205/3365; A61M 2205/502; A61M 2205/581; A61M 2205/582; A61M 2205/583

(57) ABSTRACT

Embodiments herein relate to flow control devices for cancer treatment systems and related methods. In an embodiment, a fluid flow control device for a cancer therapy system is included having a receiver housing, wherein the receiver housing can be configured to interface with a fluid delivery device. The fluid flow control device can also include a rotational plunger. The rotational plunger can include indicia, such as markings, on an end of the rotational plunger. The rotational plunger can be configured to engage with the receiver housing and rotation of the rotational plunger causes a plunger of the syringe to be depressed. Other embodiments are also included herein.

This application claims the benefit of U.S. Provisional Application No. 63/546,367, filed Oct. 30, 2023, the content of which is herein incorporated by reference in its entirety.

### FIELD

Embodiments herein relate to flow control devices for cancer treatment systems and related methods.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division. Surgery is a common first-line therapy for many cancerous tumors. However, not every tumor can be surgically removed.

### SUMMARY

Embodiments herein relate to flow control devices for cancer treatment systems and related methods. In a first aspect, a fluid flow control device for a cancer therapy system is included having a receiver housing, wherein the receiver housing can be configured to interface with a fluid delivery device. The fluid flow control device can also include a rotational plunger. The rotational plunger can include indicia, such as markings, on an end of the rotational plunger. The rotational plunger can be configured to engage with the receiver housing and rotation of the rotational plunger causes a plunger of the syringe to be depressed.

In a second aspect, in addition to one or more of the preceding or following aspects, or in the alternative to some aspects, the indicia can be configured to visually distinguish rotations of the rotational plunger.

In a third aspect, in addition to one or more of the preceding or following aspects, or in the alternative to some aspects, the fluid delivery device can be a syringe.

In a fourth aspect, in addition to one or more of the preceding or following aspects, or in the alternative to some aspects, the syringe can be from 10 ml to 100 ml in size.

In a fifth aspect, in addition to one or more of the preceding or following aspects, or in the alternative to some aspects, the fluid flow control device can further include a display screen, and control circuitry, wherein the control circuitry can be configured to cause the display screen to display an image with a rotating visual timing element.

In a sixth aspect, in addition to one or more of the preceding or following aspects, or in the alternative to some aspects, a target rotation speed of the rotational plunger can be one rotation per minute.

In a seventh aspect, in addition to one or more of the preceding or following aspects, or in the alternative to some aspects, the control circuitry can be configured to calculate a target rotation speed of the rotational plunger based on a desired fluid flow rate.

In an eighth aspect, in addition to one or more of the preceding or following aspects, or in the alternative to some aspects, the desired fluid flow rate can be from 3 ml/min to 20 ml/min.

In a ninth aspect, in addition to one or more of the preceding or following aspects, or in the alternative to some aspects, the image with a rotating visual timing element can include a clock face.

In a tenth aspect, in addition to one or more of the preceding or following aspects, or in the alternative to some aspects, the image with a rotating visual timing element can include a rotating arrow.

In an eleventh aspect, in addition to one or more of the preceding or following aspects, or in the alternative to some aspects, the display screen can be attached to the receiver housing.

In a twelfth aspect, in addition to one or more of the preceding or following aspects, or in the alternative to some aspects, the display screen can be physically separate from other components of a fluid flow control device, but in signal communication therewith.

In a thirteenth aspect, in addition to one or more of the preceding or following aspects, or in the alternative to some aspects, the fluid flow control device can be configured to receive an input from a system user or a separate device regarding a desired rotational speed of the rotational plunger and/or a desired fluid flow rate. In a fourteenth aspect, in addition to one or more of the preceding or following aspects, or in the alternative to some aspects, the rotational plunger further can include threads disposed thereon.

In a fifteenth aspect, in addition to one or more of the preceding or following aspects, or in the alternative to some aspects, the rotational plunger further can include a control knob, wherein the control knob can be disposed on an end of the rotational plunger.

In a sixteenth aspect, in addition to one or more of the preceding or following aspects, or in the alternative to some aspects, the rotational plunger can include a rotating handle.

In a seventeenth aspect, in addition to one or more of the preceding or following aspects, or in the alternative to some aspects, the receiver housing includes a cutaway portion and/or a transparent portion to allow viewing of markings on the fluid delivery device while the receiver housing interfaces with the fluid delivery device. In an eighteenth aspect, in addition to one or more of the preceding or following aspects, or in the alternative to some aspects, the rotational plunger can be configured to only turn in one direction.

In a nineteenth aspect, in addition to one or more of the preceding or following aspects, or in the alternative to some aspects, the rotational plunger can be configured so that rotation of the rotational plunger causes a plunger of a fluid delivery device to be depressed without rotating the plunger of the syringe.

In a twentieth aspect, in addition to one or more of the preceding or following aspects, or in the alternative to some aspects, a maximum travel distance of the rotational plunger can be less than a maximum depressed travel distance of a plunger of the fluid delivery device so that the rotational plunger does not cause the plunger of the fluid delivery device to become fully depressed.

In a twenty-first aspect, in addition to one or more of the preceding or following aspects, or in the alternative to some aspects, the fluid flow control device can be configured to generate sounds and/or tactile vibrations as the rotational plunger is rotated.

In a twenty-second aspect, in addition to one or more of the preceding or following aspects, or in the alternative to some aspects, the fluid flow control device can be configured to generate timing sounds to guide the device user to achieve a target rotational speed of the rotational plunger.

In a twenty-third aspect, in addition to one or more of the preceding or following aspects, or in the alternative to some aspects, the rotational plunger further can include a slip clutch mechanism, wherein the slip clutch mechanism can be configured to limit a maximum amount of force generated by the rotational plunger on the fluid delivery device.

In a twenty-fourth aspect, in addition to one or more of the preceding or following aspects, or in the alternative to some aspects, the device can further include a rotating cover, wherein the rotating cover can be configured to rotate with respect to the receiver housing.

In a twenty-fifth aspect, in addition to one or more of the preceding or following aspects, or in the alternative to some aspects, the device further can include a stop member, wherein the stop member can be configured to prevent rotation of the rotating cover beyond a specific point.

In a twenty-sixth aspect, a method of controlling a flow rate of a fluid delivery device can be included. The method can include engaging a fluid flow control device with the fluid delivery device, rotating a rotational plunger of the fluid flow control device, and providing a visual cue for a target rotational speed of the rotational plunger.

In a twenty-seventh aspect, in addition to one or more of the preceding or following aspects, or in the alternative to some aspects, providing a visual cue for a target rotational speed of the rotational plunger includes causing a display screen to display an image with a rotating timing element.

In a twenty-eighth aspect, in addition to one or more of the preceding or following aspects, or in the alternative to some aspects, the method can further include calculating a target rotation speed of the rotational plunger based on a desired fluid flow rate.

In a twenty-ninth aspect, in addition to one or more of the preceding or following aspects, or in the alternative to some aspects, the method can further include receiving an input from a system user or a separate device regarding a desired rotational speed of the rotational plunger and/or a desired fluid flow rate.

In a thirtieth aspect, in addition to one or more of the preceding or following aspects, or in the alternative to some aspects, the method can further include generating sounds and/or tactile vibrations as the rotational plunger can be rotated.

In a thirty-first aspect, in addition to one or more of the preceding or following aspects, or in the alternative to some aspects, the method can further include generating timing sounds to guide the device user to achieve a target rotational speed of the rotational plunger.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of components of a cancer therapy system in accordance with various embodiments herein.

FIG. 2 is a schematic view of a fluid flow control device and a fluid delivery device in accordance with various embodiments herein.

FIG. 3 is a schematic view of a portion of a fluid flow control device in accordance with various embodiments herein.

FIG. 4 is a schematic view of components of a fluid flow control device or system in accordance with various embodiments herein.

FIG. 5 is a schematic view of a control knob of a fluid flow control device in accordance with various embodiments herein.

FIG. 6 is a schematic view of a portion of a fluid flow control device interfacing with a plunger of a fluid delivery device in accordance with various embodiments herein.

FIG. 7 is a schematic view of a portion of a fluid flow control device interfacing with a plunger of a fluid delivery device in accordance with various embodiments herein.

FIG. 8 is a schematic view of a portion of a fluid flow control device in accordance with various embodiments herein.

FIG. 9 is a schematic view of a fluid flow control device and fluid delivery device in accordance with various embodiments herein.

FIG. 10 is a schematic view of a fluid flow control device and fluid delivery device in accordance with various embodiments herein.

FIG. 11 is a schematic view of a fluid flow control device and fluid delivery device in accordance with various embodiments herein.

FIG. 12 is a flowchart of operations of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, surgery is a common first-line therapy for many cancerous tumors. However, not every tumor can be surgically removed. Another approach to treating some types of cancer can include delivering radiation to cancerous tissue to destroy cancerous cells therein. In one approach, microspheres such as small glass microspheres that have Y-90 mixed into the glass can be deposited in or near a cancerous tumor and beta radiation emitting therefrom can damage DNA of the cancerous cells inducing apoptosis and cell cycle arrest.

Delivery of microspheres can be performed using a catheter, such as a microcatheter placed in vasculature leading to a tumor, such as an artery supplying blood to the tumor. The microspheres can be combined with a fluid, such as saline, to form a suspension that is then directed through the catheter to the site of deposition.

In some approaches, a clinician must utilize a syringe or similar device to deliver the fluid to be mixed with the microspheres. The clinician advances the plunger of the syringe at a desired pressure/speed resulting in a particular flow rate of fluid through the system for a particular amount of time to a achieve a target volume of suspension delivered out of the catheter and into the patient. However, it is tedious for system users to manage aspects of this process. For example, it is difficult for system users to accurately determine the actual flow rate achieved (such as between 5 ml/min and 20 ml/min, or within other flow rate bounds).

Systems and methods herein can be used to assist a device user in achieving a desired flow rate from fluid delivery device such as a syringe. In an embodiment, a fluid flow control device for a cancer therapy system is included having a receiver housing, wherein the receiver housing can be configured to interface with a fluid delivery device. The fluid flow control device can also include a rotational plunger. The rotational plunger can include indicia, such as markings, on an end of the rotational plunger. The rotational plunger can be configured to engage with the receiver housing and rotation of the rotational plunger causes a plunger of the syringe to be depressed. The indicia can be configured to visually distinguish rotations of the rotational plunger. The device user can reference a visual timing guide, such as a (real or virtual) second hand of a clock, a rotating arrow, or the like, and match the speed of rotations of the rotational plunger with the speed of rotations illustrated by the visual timing guide to result in a depression speed of the plunger of the fluid delivery device that achieves a target flow rate from the fluid delivery device.

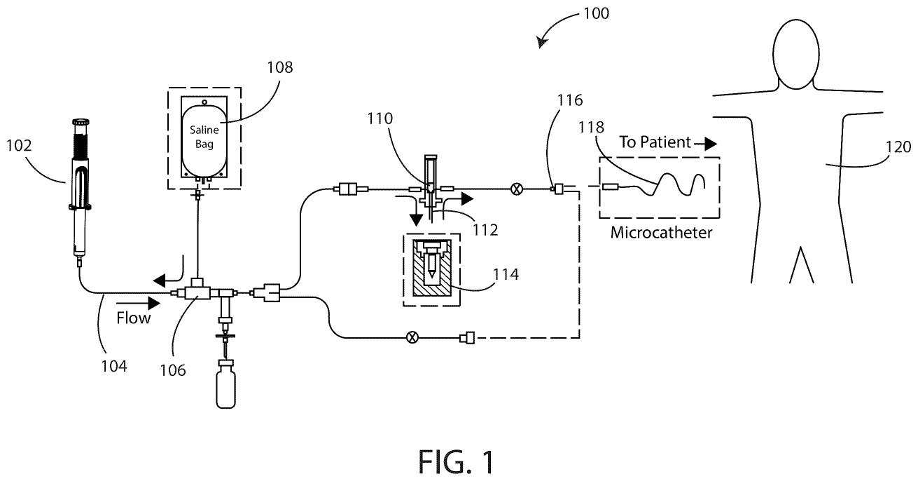

Referring now to FIG. 1, a diagram of a cancer treatment system 100 is shown in accordance with various embodiments herein. Major parts of the cancer treatment system 100 include a therapeutic fluid delivery device assembly 102 including a fluid delivery device engaged with a fluid flow control device described in more detail below. The cancer treatment system 100 can also include a fluid supply tube 104 and a flow control valve 106. The cancer treatment system 100 also includes a saline supply reservoir 108. The cancer treatment system 100 also includes a fluid injection and suspension withdrawal device 110. The cancer treatment system 100 also includes a radioactive microsphere supply reservoir 114. The cancer treatment system 100 also includes an outflow port 116. The cancer treatment system 100 also includes a microcatheter 118. FIG. 1 also shows a patient 120 in which the microcatheter 118 can be inserted to deliver the therapeutic suspension of microspheres.

In use, (omitting some possible operations for ease of explanation) the clinician or other system user can utilize the fluid flow control device to pull back on the plunger (or similar element) of a fluid delivery device causing fluid (such as saline) to be withdrawn from the saline supply reservoir 108, through the flow control valve 106 and the fluid supply tube 104, and into the fluid delivery device. Alternatively, the fluid delivery device may already be filled with a fluid at the beginning of the procedure.

Then, using the fluid flow control device, the clinician or other system user can depress the plunger causing fluid to flow from the therapeutic fluid delivery device, through the fluid supply tube 104, through the flow control valve 106, and into the fluid injection and suspension withdrawal device 110. The fluid injection and suspension withdrawal device 110 can be in fluid communication with the radioactive microsphere supply reservoir 114 and can direct a flow of fluid into the radioactive microsphere supply reservoir 114 coming from the therapeutic fluid delivery device assembly 102 such as through one of a pair of needles, cannulas, or tubes 112. The fluid can become mixed with microspheres in the radioactive microsphere supply reservoir 114 forming a suspension which can then exit via the fluid injection and suspension withdrawal device 110 via another needle, cannula, or tube 112 and through tubing and out of the outflow port 116 and into the microcatheter 118 and into a desired site of the patient 120. It will be appreciated that various other operations can also be performed including, but not limited to, system priming, bubble removal, one or more flushing operations, and the like.

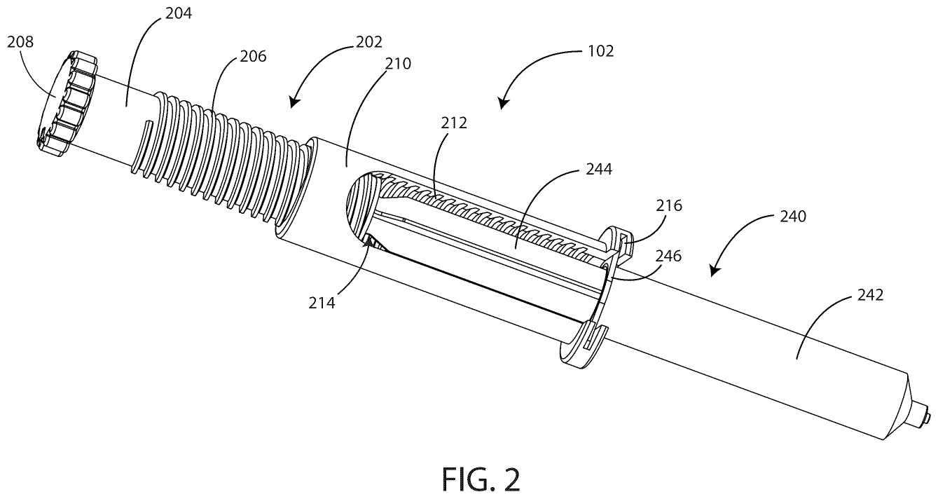

Referring now to FIG. 2, a schematic view of a fluid delivery device assembly 102 is shown including a fluid flow control device 202 and a fluid delivery device 240. The fluid flow control device 202 includes a rotational plunger 204. The fluid flow control device 202 also includes a receiver housing 210. The rotational plunger 204 can be configured to fit within the receiver housing 210 such that rotation of the rotational plunger 204 causes a portion of the rotational plunger 204 or a component connected thereto to be pushed into the receiver housing 210. In various embodiments, the rotational plunger 204 includes threads 206 disposed thereon. The receiver housing 210 can include complementary threads 212 that can engage with the threads 206 of the rotational plunger 204 to facilitate translating rotation of the of the rotational plunger 204 into linear motion driving the rotational plunger 204 or a component attached thereto further into the receiver housing 210 or farther out of the receiver housing 210 depending on the direction of rotation.

While the use of threads are one approach to allow for the translation of rotation into linear motion, there are other approaches that can be used and are contemplated herein. By way of example, in some embodiments, one or more pins or projections can be disposed on the rotation plunger that can fit within and move within one or more helical slots on the receiver housing 210. Other approaches contemplated herein can include slider-crank mechanisms, rack and pinion mechanisms, belts, pulleys, and the like.

In this example, the fluid delivery device 240 includes a barrel 242, inside of which a fluid can be held. The fluid delivery device 240 also includes a plunger 244. As the plunger 244 is depressed into the barrel 242, pressure is increased causing fluid to flow out of the fluid delivery device 240. In this example, the fluid delivery device 240 also includes a flange 246, which can be used as a finger rest when the fluid delivery device 240 is directly manipulated, but can be utilized herein as a point of engagement with the fluid flow control device 202. For example, the flange 246 can fit within a flange slot 216 of the receiver housing 210. The receiver housing 210 can also include an access slot 214 to facilitate fitting the fluid delivery device 240 together with the fluid flow control device 202. In some embodiments, the receiver housing 210 can include a cutaway portion and/or a transparent portion to allow viewing of markings on the fluid delivery device 240 (such as volume markings) while the receiver housing 210 interfaces with the fluid delivery device 240.

As referenced previously, the rotational plunger 204 can be configured to engage with a receiver housing 210. Rotation of the rotational plunger 204 causes the rotational plunger 204 to move with respect to the receiver housing. When the fluid flow control device 202 is engaged with the fluid delivery device 240, rotation of a rotational plunger 204 causes a plunger 244 of the fluid delivery device 240 to be depressed. The rotational plunger 204 can also include a control knob 208 or dial or similar element. In various embodiments, the control knob 208 can be disposed on an end of a rotational plunger 204. The control knob 208 can allow for easy manual manipulation of the rotational plunger 204, such as rotating the rotational plunger 204 in one direction or another. In some embodiments, a rotating handle can be include instead of or in addition to the control knob 208.

In various embodiments, the rotational plunger 204 can be configured to only turn in one direction, but in other embodiments the rotational plunger 204 can be configured to turn in both directions. In some embodiments, a maximum travel distance of the rotational plunger 204 can be configured to be less than a maximum depressed travel distance of a plunger 244 of the fluid delivery device 240. This can be accomplished in various ways, such as ending the complementary threads 212 of the receiver housing 210 a point preventing the rotational plunger 204 from being advanced any further. In this manner, the rotational plunger 204 can be configured so that it does not cause the plunger 244 of the fluid delivery device 240 to become fully depressed.

In various embodiments, the fluid delivery device 240 can take the form of a syringe, however, other fluid delivery devices are also contemplated herein. The syringe can be of various sizes. In some embodiments, the syringe can be from 10 ml to 100 ml in size.

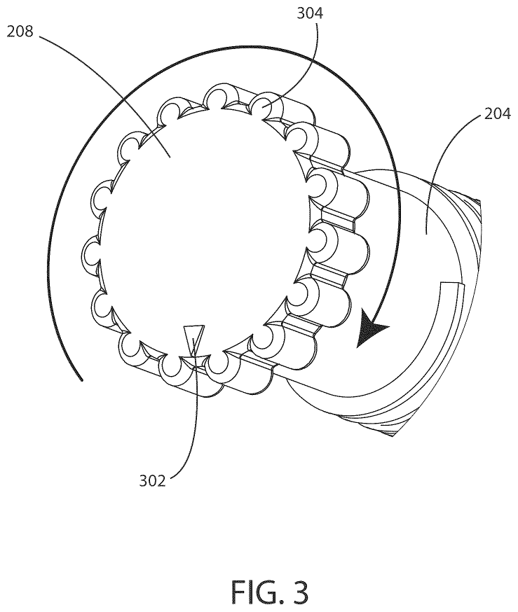

Referring now to FIG. 3, a schematic view of a portion of a fluid flow control device is shown in accordance with various embodiments herein. In specific, a rotational plunger 204 including a control knob 208 or dial. The control knob 208 can include indicia 302 and grip projections 304 disposed thereon. In various embodiments, the indicia 302 can be disposed on an end of a rotational plunger 204. In various embodiments, the indicia 302 are configured to visually indicate/distinguish rotations of a rotational plunger 204. As used herein, reference to “indicia” can refer to a single marking or visual element or multiple markings or visual elements. It will be appreciated that the indicia can take various forms. In some embodiments, the indicia can take the form of a raised projection or other visible physical element. In some embodiments, the indicia can take the form of ink or other marking materials disposed on or in the control knob 208. In some embodiments, the indicia can take the form of letters, numerals, symbols, shapes, and the like. In some embodiments, the indicia can be a different color than portions of the control knob 208 to achieve an easily perceived visual contrast.

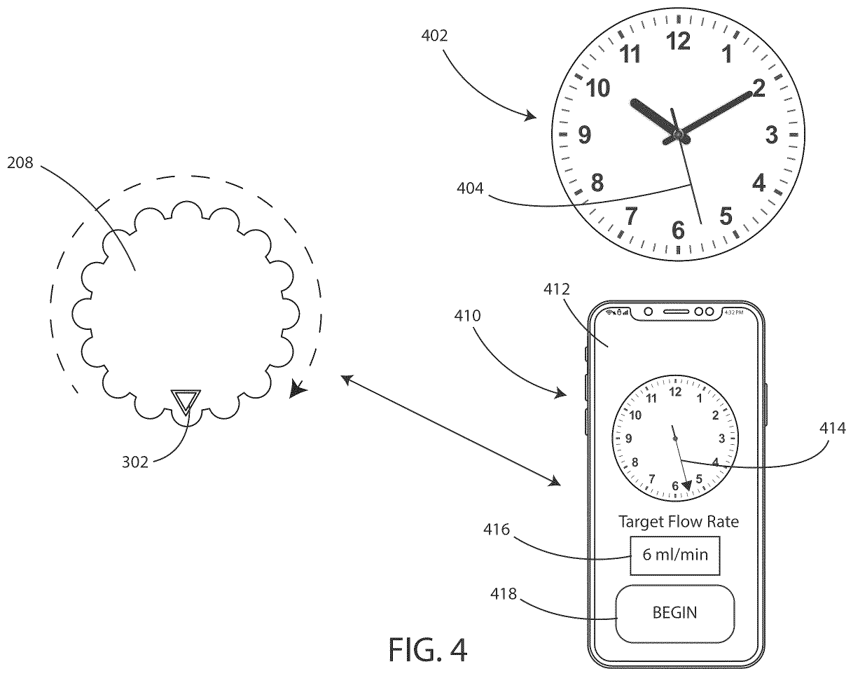

Referring now to FIG. 4, a schematic view of components of a fluid flow control device or system is shown in accordance with various embodiments herein. In specific, FIG. 4 shows a control knob 208 which can be attached to (directly or indirectly) or otherwise associated with a rotational plunger. In this example, the control knob 208 also includes indicia 302 disposed thereon.

FIG. 4 also shows a clock face 402, which can be real or virtual. The clock face 402 includes a rotating second hand 404. Rotation of the second hand 404 occurs at a speed of one rotation per minute. FIG. 4 also shows a separate device 410. The separate device 410 includes a display screen 412 showing various elements thereon. In various embodiments, the separate device 410 and/or other components of the system herein can include control circuitry. In some embodiments, the control circuitry can be configured to cause the display screen 412 to display an image with a visual timing guidance element. In some embodiments, the control circuitry can be configured to cause the display screen 412 to display an image with a rotating timing element. For example, the display screen 412 can be configured to show a rotating arrow 414.

The display screen 412 can also be configured to show a target flow rate 416. The separate device 410 also includes a user interface button 418. It will be appreciated that various other user interface objects (buttons, sliders, drop-downs, etc.) can be shown on the display screen 412.

In various embodiments, the fluid flow control device 202 can be configured to receive an input from a system user or a separate device 410 regarding a desired rotational speed of the rotational plunger 204 and/or a desired fluid flow rate. For example, in some embodiments, the separate device 410 or the system herein can be configured to receive user input regarding a target flow rate 416 such as to set, or adjust a target flow rate. In various embodiments, the control circuitry can be configured to calculate a target rotation speed of the rotational plunger 204 based on a target fluid flow rate. For example, if the full rotation of the rotational plunger 204 translates to a certain distance (D) of linear motion of the rotational plunger 204 or another element and if the cross-sectional area of the barrel 242 of the fluid delivery device 240 is (A), then each rotation of the rotational plunger 204 results in a volume of fluid moving in or out of the fluid delivery device 240 of (V/R), wherein V/R=D×A. If the target flow rate is (FR), then a target rotation speed can be calculated according to the formula FR=V/R×v, wherein (v) is rotations per unit time (such as rotations per minute).

In various embodiments, a target rotation speed of the rotational plunger 204 can be one rotation per minute, such that the target rotation speed matches the rotation speed of a second hand of a clock. However, the target rotation speed can be any particular speed and can be calculated, accessed from memory, or received as input. In various embodiments, the desired fluid flow rate can be from 3 ml/min to 20 ml/min.

In various embodiments, the display screen can be physically separate from other components of a fluid flow control device 202, but in signal communication (wired or wireless) therewith. For example, in the embodiment shown in FIG. 4, the display screen 412 is a part of the separate device 410. However, in various embodiments, the display screen 412 can be attached to the receiver housing 210 instead of being part of a separate device 410.

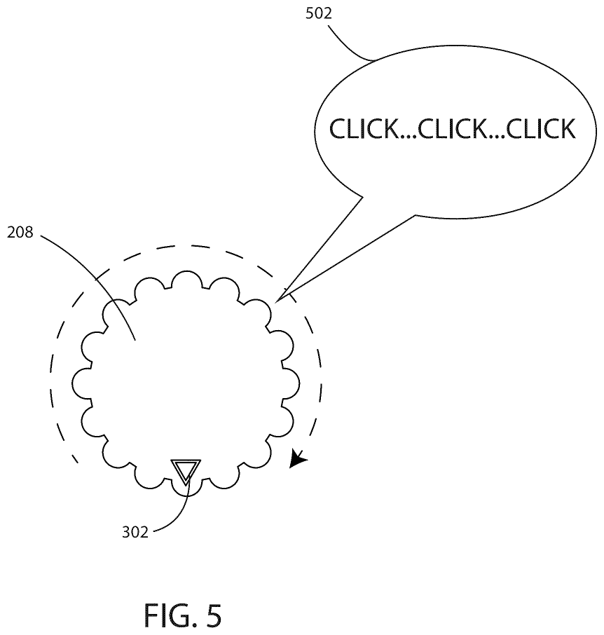

In some embodiments, the fluid flow control device can be configured to generate sounds and/or tactile vibrations as the rotational plunger is rotated. Referring now to FIG. 5, a schematic view of a control knob 208 of a fluid flow control device is shown in accordance with various embodiments herein. As before, the rotational plunger also includes indicia 302. In this example, the fluid flow control device generates sounds (such as a clicking, pinging, or beeping sound or the like) as the control knob 208 is rotated. For example, in some embodiments the device can be configured so that there are 60 sounds (as discrete sounds or as identifiable elements within a pattern of sounds) emitted per rotation of the rotational plunger. In some embodiments, a different number of sounds can be emitted per rotation, such as 1, 2, 3, 4, 5, 10, 15, 20, 30, 40, 50, 60, 90, 120, or more, or a number falling within a range between any of the foregoing. In some embodiments, the fluid flow control device can, in addition or in place of generating sounds, generate tactile vibrations.

In some embodiments, generating sounds and/or tactile vibrations can be accomplished mechanically or via electrical means. In some embodiments, generating sounds and/or tactile vibrations can be accomplished mechanically using a mechanism such as a click arm interfacing with a nub or toothed mechanical part, wherein the click arm is connected to the control knob 208 or other parts of the rotational plunger in such a manner that it is caused to sweep over a series of nubs or teeth generating an audible click each time it does. Such a mechanism can generate sound and a tactile vibration simultaneously. In some embodiments, such a mechanism or the like can create slight increases in the force required to rotate the rotational plunger at discrete points of rotation such that each unit or subunit of rotation (1, 2, 3, 4, 5, 6, 12, 18, 36, 60, 120, 180, or 360 degrees, or an amount falling within a range between any of the foregoing) can be felt by a device user rotating the rotational plunger even in the absence of audible click or sounds. In some embodiments, a powered sound generator can be used such as a speaker along with a sensor to trigger when the speaker should make sound. The sensor can be connected to the control knob 208 or other parts of the rotational plunger to detect rotation of the same.

In some embodiments, the fluid flow control device can be configured to generate timing sounds to guide the device user to achieve a target rotational speed of the rotational plunger. For example, the device or system herein can include a metronome component to emit sounds and/or vibrations at a particular pace. The metronome component can produce such sounds at a fixed pace or interval corresponding to a target rate of rotation. A metronome component can be used independently or in combination with a rotational plunger that is configured to generate sounds and/or tactile vibrations. When used in combination, the metronome component can allow the device user to easily match actual rotational plunger rotation speed with the target rotational speed as represented by the metronome cadence.

It can be desirable for the rotational plunger to be configured so that rotation of the rotational plunger causes a plunger of the fluid delivery device to be depressed without rotating and/or applying rotational force onto the plunger of the syringe. This is particularly valuable where the plunger of the fluid delivery device is not designed to be turned as it is depressed. Otherwise, if rotational force is provided to the plunger of the fluid delivery device, it may cause the plunger of the fluid delivery device to twist and/or otherwise distort undesirably. As such, in some embodiments bearings such as turntable bearings or a similar mechanical element can be used on an end of the rotational plunger with a contact plate that makes contact with the plunger of the fluid delivery device, allowing the contract plate to remain rotationally fixed while the rotational plunger rotates. This can allow linear motion of the rotational plunger to depress the plunger of the fluid delivery device without the rotational force of the plunger being applied to the plunger of the fluid delivery device. Other mechanical mechanisms to prevent rotational force of the rotational plunger from being transferred to the plunger of the fluid delivery device are also contemplated herein.

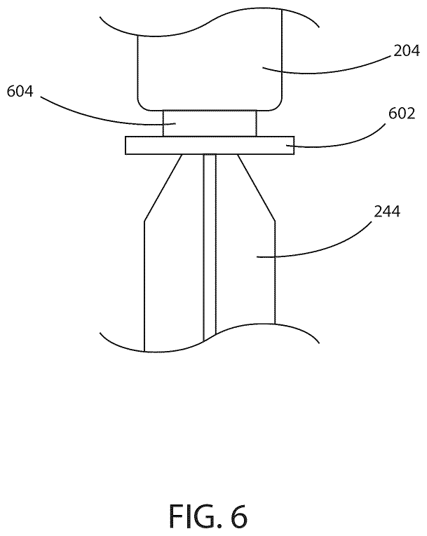

Referring now to FIG. 6, a schematic view of a portion of a fluid flow control device interfacing with a plunger 244 of a fluid delivery device is shown in accordance with various embodiments herein. The rotational plunger 204 of a fluid flow control device along with the plunger 244 of the fluid delivery device. In this embodiment, the plunger 244 includes a plunger head 602 and a rotational bearing/contact plate assembly 604. The rotational bearing/contact plate assembly 604 allows for the rotational plunger 204 to apply linear force onto the plunger 244 without conveying rotational force onto the plunger 244 which may otherwise cause the same to twist. Thus, in various embodiments, the rotational plunger 204 can be configured so that rotation of the rotational plunger 204 causes a plunger 244 of the syringe to be depressed without rotating the plunger 244 of the syringe.

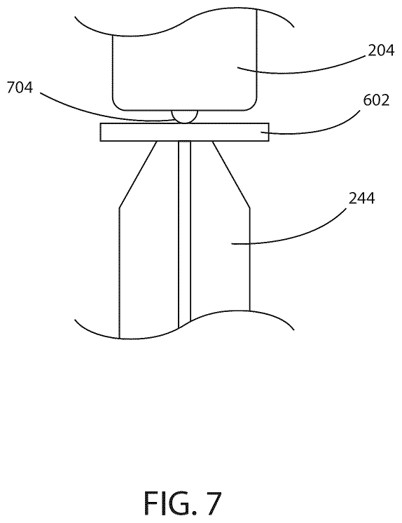

Referring now to FIG. 7, a schematic view of a portion of a fluid flow control device interfacing with a plunger 244 of a fluid delivery device is shown in accordance with various embodiments herein. FIG. 7 is generally similar to FIG. 6. However, instead of a rotational bearing/contact plate assembly, in the embodiment of FIG. 7, a nub 704 or raised pivot point is included. The nub 704 can contact the plunger head 602 and apply linear force thereto but not convey rotational force to the plunger head 602 because it only contacts the plunger head 602 at a small point. Various other similar mechanical structures to apply linear force without conveying rotational force are also contemplated herein.

The mechanical advantage associated with rotating a rotational plunger generating torque that is translated into a linear force to depress a plunger can be substantial depending on various aspects such as the axial distance between screw threads, the diameter of control knob or lever to turn the rotational plunger, and the like. In some embodiments, a mechanism can be included to limit the total amount of torque that can be applied to the rotational plunger, such as based on a device user manually turning the rotational plunger. For example, a slip clutch mechanism, a torque overload clutch, a torque limiter (such as a mechanical friction torque limiter, a magnetic torque limiter, or the like), or a similar element can be used to limit the total amount of torque that can be applied to the rotational plunger.

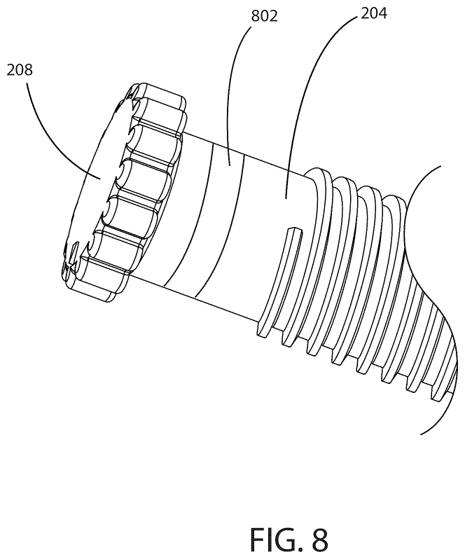

Referring now to FIG. 8, a schematic view of a portion of a fluid flow control device is shown in accordance with various embodiments herein. As before, the fluid flow control device includes a rotational plunger 204 with a control knob 208 disposed thereon. In this embodiment, the rotational plunger 204 includes a slip clutch mechanism 802. In various embodiments, the slip clutch mechanism 802 can be configured to limit a maximum amount of force (torque) that can be transferred from a manually manipulated element such as the control knob 208 to the rest of the rotational plunger 204 and therefore limit a maximum amount of force applied to a plunger of a fluid delivery device 240 by the rotational plunger 204.

It can be desirable to accommodate fluid delivery devices of various sizes. Further, in some scenarios, it can be useful to shield the fluid delivery device from the device user.

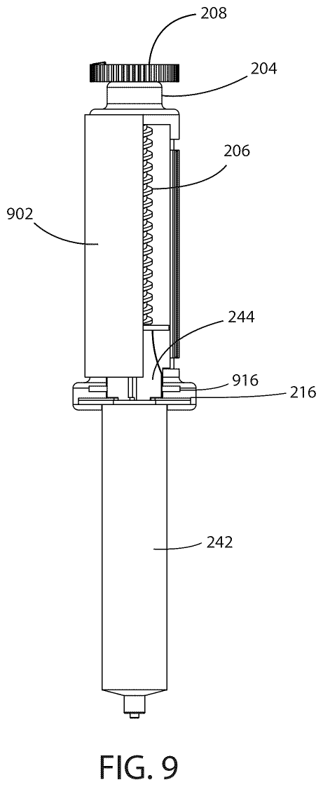

Referring now to FIG. 9, a schematic view is shown of a fluid flow control device and fluid delivery device in accordance with various embodiments herein. In this view, components are shown including rotational plunger 204, threads 206, control knob 208, flange slot 216, barrel 242, and plunger 244. In this embodiment, a second flange slot 916 can also be included. The second flange slot 916 can be used to accommodate a fluid delivery device of a different size. This can enable the device user to choose the correct fluid delivery device size needed for a given operation.

In the embodiment of FIG. 9, a cover 902 (or shield) is also included. The cover 902 can serve to hold the fluid delivery device in place so it cannot fallout of the device. In some embodiments, the cover 902 can be made of a softer textured material (softer durometer value) that makes it more comfortable for the device user to hold and eliminates the hand from being pinched in the device. The cover 902 can rotate to an open position where the fluid delivery device can be inserted into the fluid flow control device and can then rotate to a closed position.

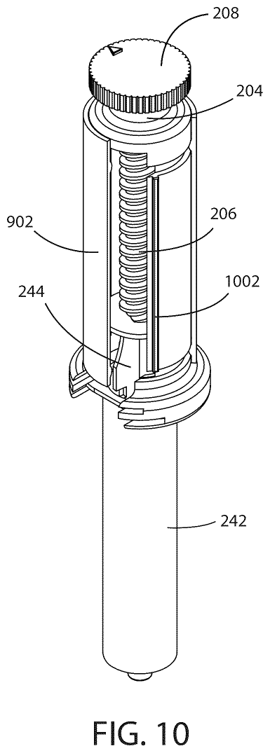

Referring now to FIG. 10, a schematic view is shown of a fluid flow control device and fluid delivery device in accordance with various embodiments herein. In this view, components are shown including rotational plunger 204, threads 206, control knob 208, barrel 242, and plunger 244. In this example, the device has a cover 902 along with a stop 1002, so the cover 902 will not continue to rotate beyond the point of the stop 1002 allowing the user to hold the device as the control knob 208 is turned.

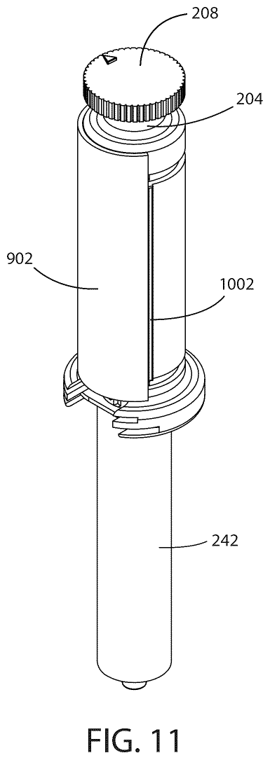

Referring now to FIG. 11, a schematic view is shown of a fluid flow control device and fluid delivery device in accordance with various embodiments herein. In this view, components are shown including rotational plunger 204, threads 206, control knob 208, barrel 242, and plunger 244. In this example, the device has a cover 902 that has now been rotated into a fully closed position wherein it rests against the stop 1002, preventing any further rotation.

### Methods

Many different methods are contemplated herein, including, but not limited to, methods of making, methods of using, methods of controlling a fluid flow rate of a cancer treatment device, and the like. Aspects of system/device operation described elsewhere herein can be performed as operations of one or more methods in accordance with various embodiments herein.

In an embodiment, a method of controlling a flow rate of a fluid delivery device is included. The method can include engaging a fluid flow control device with the fluid delivery device, rotating a rotational plunger of the fluid flow control device, and providing a visual cue for a target rotational speed of the rotational plunger. In an embodiment of the method, providing a visual cue for a target rotational speed of the rotational plunger comprises causing a display screen to display an image with a rotating timing element. In an embodiment, the method can further include calculating a target rotation speed of the rotational plunger based on a desired fluid flow rate.

In an embodiment, the method can further include receiving an input from a system user or a separate device regarding a desired rotational speed of the rotational plunger and/or a desired fluid flow rate.

In an embodiment, the method can further include generating sounds and/or tactile vibrations as the rotational plunger is rotated. In an embodiment, the method can further include generating timing sounds to guide the device user to achieve a target rotational speed of the rotational plunger.

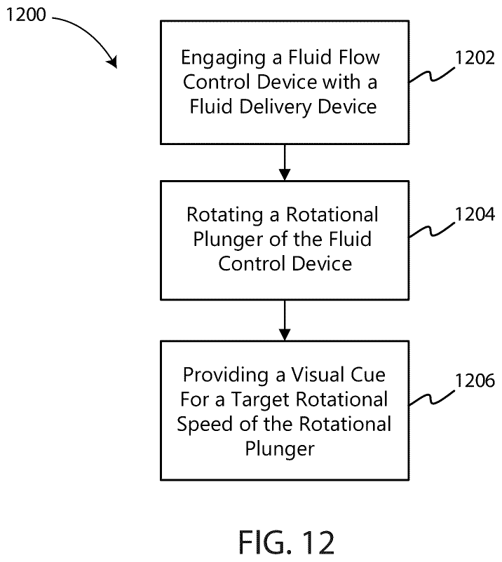

Referring now to FIG. 12, a flowchart of operations of a method is shown in accordance with various embodiments herein. FIG. 12 shows a method of controlling a flow rate of a fluid delivery device 1200. The method of controlling a flow rate of a fluid delivery device 1200 includes an engaging a fluid flow control device with a fluid delivery device 1202. The method of controlling a flow rate of a fluid delivery device 1200 also includes a rotational plunger of a fluid flow control device 1204. The method of controlling a flow rate of a fluid delivery device 1200 also includes a providing a visual cue for a target rotational speed of a rotational plunger 1206.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A fluid flow control device for a cancer therapy system comprising:
a receiver housing, wherein the receiver housing is configured to interface with a fluid delivery device; and
a rotational plunger, the rotational plunger comprising indicia, wherein the indicia are disposed on an end of the rotational plunger;
wherein the rotational plunger is configured to engage with the receiver housing; and
wherein rotation of the rotational plunger causes a plunger of the syringe to be depressed.

2. The fluid flow control device of claim 1, the fluid delivery device comprising a syringe.

3. The fluid flow control device of claim 1, further comprising:
a display screen; and
control circuitry, wherein the control circuitry is configured to cause the display screen to display an image with a rotating timing element.

4. The fluid flow control device of claim 3, wherein the control circuitry is configured to calculate a target rotation speed of the rotational plunger based on a desired fluid flow rate.

5. The fluid flow control device of claim 3, the image with a rotating timing element comprising a clock face.

6. The fluid flow control device of claim 3, the image with a rotating timing element comprising a rotating arrow.

7. The fluid flow control device of claim 1, the rotational plunger further comprising a knob, wherein the knob is disposed on an end of the rotational plunger.

8. The fluid flow control device of claim 1, wherein the receiver housing includes a cutaway portion and/or a transparent portion to allow viewing of markings on the fluid delivery device while the receiver housing interfaces with the fluid delivery device.

9. The fluid flow control device of claim 1, wherein the rotational plunger is configured to only turn in one direction.

10. The fluid flow control device of claim 1, wherein the rotational plunger is configured so that rotation of the rotational plunger causes a plunger of the syringe to be depressed without rotating the plunger of the syringe.

11. The fluid flow control device of claim 1, wherein a maximum travel distance of the rotational plunger is less than a maximum depressed travel distance of a plunger of the fluid delivery device so that the rotational plunger does not cause the plunger of the fluid delivery device to become fully depressed.

12. The fluid flow control device of claim 1, wherein the fluid flow control device is configured to generate sounds and/or tactile vibrations as the rotational plunger is rotated.

13. The fluid flow control device of claim 12, wherein the fluid flow control device is configured to generate timing sounds to guide the device user to achieve a target rotational speed of the rotational plunger.

14. The fluid flow control device of claim 1, the rotational plunger further comprising a slip clutch mechanism, wherein the slip clutch mechanism is configured to limit a maximum amount of force generated by the rotational plunger on the fluid delivery device.

15. The fluid flow control device of claim 1, further comprising a rotating cover, wherein the rotating cover is configured to rotate with respect to the receiver housing.

16. The fluid flow control device of claim 15, further comprising a stop member, wherein the stop member is configured to prevent rotation of the rotating cover beyond a specific point.

17. A method of controlling a flow rate of a fluid delivery device comprising:
engaging a fluid flow control device with the fluid delivery device;
rotating a rotational plunger of the fluid flow control device; and
providing a visual cue for a target rotational speed of the rotational plunger.

18. The method of claim 17, wherein providing a visual cue for a target rotational speed of the rotational plunger comprises causing a display screen to display an image with a rotating timing element.

19. The method of claim 17, further comprising generating sounds and/or tactile vibrations as the rotational plunger is rotated.

20. The method of claim 17, further comprising generating timing sounds to guide the device user to achieve a target rotational speed of the rotational plunger.
